(19) United States
(12) Patent Application Publication
(10) Pub. No.: US 2026/0137433 A1
(43) Pub. Date: May 21, 2026

(54) METHODS AND DEVICES FOR SEMI-RIGID BONE FIXATION

(71) Applicant: DePuy Synthes Products, Inc., Raynham, MA (US)

(72) Inventor: Todd J. KENT, Raynham, MA (US)

(73) Assignee: DePuy Synthes Products, Inc., Raynham, MA (US)

(21) Appl. No.: 18/954,715

(22) Filed: Nov. 21, 2024

(51) Int. Cl. A61B 17/84; A61B 17/80

(52) U.S. Cl. CPC A61B 17/846; A61B 17/8019

(57) ABSTRACT

An example apparatus is provided for the approximation of two bones. The apparatus can include a first anchor configured for insertion into a first bone. The apparatus can include a flexible segment, and an internal mechanism configured to adjust a distance between the first bone and a second bone. The internal mechanism can include first and second components, the first component configured to engage with a distal end of the flexible segment, and the second component configured to engage with the first component and the first anchor such that the second component is movable along a length of the first anchor to cause the first component to move thereby adjusting the distance. The apparatus can include a second anchor configured to engage with the second bone and the flexible segment such that the flexible segment extends between the first component of the internal mechanism and the second anchor.

### FIELD

The present invention generally relates to methods and devices for semi-rigid fixation of bones. More specifically, certain embodiments relate to systems and methods for fixation of the distal tibia and distal fibula following an injury to the corresponding syndesmotic joint.

### BACKGROUND

A syndesmotic injury results when a traumatic injury damages the ligaments that span the gap between the distal tibia and fibula. This can be the result of a high ankle sprain, with no fracture of the fibula, or can also accompany a fibular fracture in a Weber B or Weber C fracture.

A surgeon can determine the presence of a syndesmotic injury by direct visualization of the joint or through radiographic imaging while positioning the ankle in a mortise view orientation. In either case, loads are applied to the joint in either a direct lateral load applied to the fibula or by applying an external rotation load to the foot. While the load is being applied, the relative distance between the fibula and the tibia, the fibula and the talus, and the tibia and the talus are observed to determine the level of damage sustained by the ligaments that typically hold the syndesmotic joint together.

If a syndesmotic injury is found to be present, the typical treatment involves stabilizing the fibula and tibia with respect to each other in the proper orientation and holding them there throughout the soft tissue healing period to allow the ligaments to re-attach and heal. In the event of a syndesmotic injury with a corresponding fibula fracture, this is done while also stabilizing the fibular fracture, which is usually accomplished with a small fracture plate on the lateral side of the fibula. Traditionally the method of stabilization has been to place one or more cortical screws across the syndesmosis, with the head against the lateral face of the fibula and the tip of the screw being in the middle of the tibia or in the medial cortex of the tibia.

This form of treatment provides very rigid fixation, allowing the ligaments to heal, but makes return to weight-bearing more difficult. During a standard gait, the ligaments hold the distance between the tibia and fibula fairly constant, but allow a small amount of shear motion and rotation of the fibula with respect to the tibia. The presence of the fixation screws prevents this motion and can cause discomfort and limited flexibility of the ankle joint. Typically, the surgeon prescribes a secondary surgery to remove the screws once the ligaments have healed. In some cases, a surgeon may simply recommend a return to weight-bearing when the ligaments have healed and, after a period of time of loading the screws, they will experience a fatigue failure and normal anatomical motion will be restored.

To address these rigidity issues, some methods of stabilization have been developed to include a flexible internal segment connected by a first anchor on the lateral side of the fibula and a second anchor on the medial side of the tibia. These methods, however, require a through or bore hole through the medial wall of the tibia, which not only provides an additional point of necessary recovery, but also requires a physician to access a patient from multiple sides and angles during treatment.

Furthermore, traditional methods of implanting such devices into the tibia and fibula bones can be cumbersome and inefficient, requiring numerous steps and/or pieces of equipment.

Accordingly, alternative apparatus and methods for providing semi-rigid fixation of the distal tibia and fibula following a syndesmotic injury would be useful.

### SUMMARY

The present invention is directed to apparatus and methods for stabilizing a joint between two bones (e.g., the tibia and fibula) or bone fragments, during the soft tissue healing period following a traumatic injury.

In an exemplary application, the apparatus and methods herein may be configured to provide substantially rigid tensile fixation between the tibia and fibula, or fragments thereof, while allowing the small amount of shear and rotational motion required for a standard gait. This may make it possible for patients to return to weight-bearing earlier, which may improve clinical outcomes, and/or may also reduce the number of follow-up hardware-removal surgeries.

An example apparatus is provided for the approximation of two bones. The apparatus can include a first anchor having a proximal end and a distal end, and configured for insertion into a first bone. The apparatus can include a flexible segment, and an internal mechanism configured to adjust a distance between the first bone and a second bone. The internal mechanism can include first and second components, the first component configured to engage with a distal end of the flexible segment, and the second component configured to engage with the first component and the first anchor such that the second component is movable along a length of the first anchor to cause the first component to move thereby adjusting the distance. The apparatus can include a second anchor configured to engage with the second bone and the flexible segment such that the flexible segment extends between the first component of the internal mechanism and the second anchor.

An example system is provided for the approximation of two bones. The system can include an apparatus and a delivery device configured to engage the apparatus with a first bone and a second bone. The apparatus can include a first anchor, a flexible segment, an internal mechanism, and a second anchor, the first anchor having a distal end configured for insertion into a first hole in a first bone, the internal mechanism being configured to engage the first anchor to adjust a distance between the first bone and a second bone, the second anchor being configured to engage with the second bone, and the flexible segment extending between the internal mechanism and the second anchor. The delivery device can include a first handle and a second handle, the first handle being configured to insert the first anchor into the first bone, and the second handle being configured to engage the internal mechanism with the first anchor, thereby adjusting the distance between the first and second bones.

An example method is provided for the approximation of two bones. The method can include delivering, via a first handle of a delivery device, a first anchor into a first bone. The first anchor includes an internal mechanism therein, and the internal mechanism engages with a distal end of a flexible segment that extends between the internal mechanism and a second anchor. The method can include simultaneously, via a second handle of the delivery device, engaging the second anchor with a second bone, and adjusting placement of the internal mechanism within the first anchor thereby adjusting placement of the distal end of the flexible segment within the first anchor and a distance between the first and second bones.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIG. 1 is an example apparatus for semi-rigid bone fixation, according to aspects of the present invention.

FIG. 2A is an example apparatus for semi-rigid bone fixation, according to aspects of the present invention.

FIG. 2B provides example components of an apparatus for semi-rigid bone fixation, such as the apparatus of FIG. 2A, according to aspects of the present invention.

FIGS. 3A-3E provide example components of a second anchor of an apparatus for semi-rigid bone fixation, such as the apparatus of FIG. 2A, according to aspects of the present invention.

FIGS. 4A-4D provide example first components of an apparatus for semi-rigid bone fixation, such as the apparatus of FIG. 2A, according to aspects of the present invention.

FIG. 4E provides an example second component of an apparatus for semi-rigid bone fixation, such as the apparatus of FIG. 2A, according to aspects of the present invention.

FIG. 5A is an example system for semi-rigid bone fixation, according to aspects of the present invention.

FIG. 5B is a cross-section of the system of FIG. 5A, according to aspects of the present invention.

FIG. 6 is a step in an example method for the approximation of two bones, according to aspects of the present invention.

FIG. 7 is a step in an example method for the approximation of two bones, according to aspects of the present invention.

FIG. 8 is a step in an example method for the approximation of two bones, according to aspects of the present invention.

FIG. 9 is a cross-section of components of an example system during a step in a method for the approximation of two bones, such as the step shown in FIG. 7, according to aspects of the present invention.

FIG. 10 is a step in an example method for the approximation of two bones, according to aspects of the present invention.

FIG. 11 is a step in an example method for the approximation of two bones, according to aspects of the present invention.

### DETAILED DESCRIPTION

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g., “about 70%” may refer to the range of values from 50% to 90%.

The example devices and methods of treatment described herein generally involve providing semi-rigid fixation of two bones, such as the tibia and fibula bones, by implanting an apparatus, including a first anchor, a second anchor, a flexible segment, and an internal mechanism, through the fibula bone and into only one side of the tibia bone. That is, the disclosed example devices and methods of treatment do not require a second hole be made through the medial wall of the tibia bone. Instead, an apparatus can be inserted through the fibula bone and only partially into the tibia bone. The disclosed example devices and methods of treatment thus prevent an additional point of necessary patient recovery. Additionally, the example delivery devices described herein provide for improved usability in terms of efficient delivery of fixation devices into the fibula and tibia bones.

Various example systems and methods are presented herein. Features from each example are combinable with other examples as understood by persons skilled in the pertinent art.

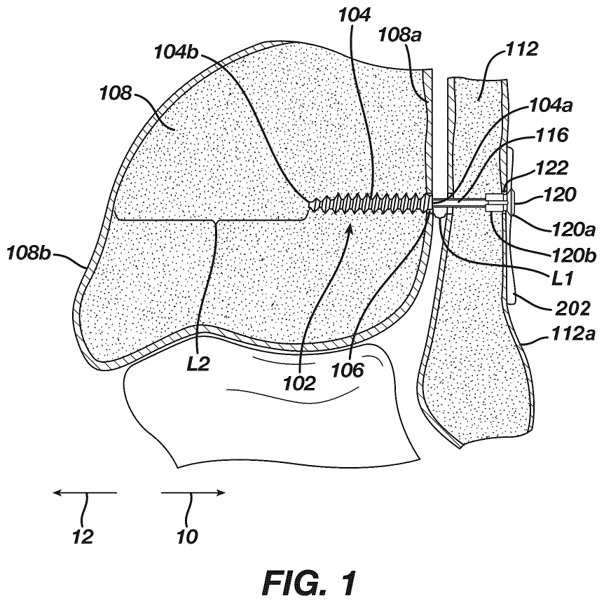

FIG. 1 is an illustration of an example environment in which an example apparatus 102 can be used for semi-rigid bone fixation. The apparatus 102 for the approximation of two bones, such as the tibia and fibula bones, can include a first anchor 104, which includes a proximal end 104a and a distal end 104b configured for insertion into a first hole 106 in a first side 108a of a first bone 108, e.g., a tibia bone. The apparatus 102 can also include a flexible segment 116, and an internal mechanism 110 configured to adjust a distance L1 between the first bone 108 and a second bone 112 (e.g., a fibula bone). As further discussed below, the internal mechanism 110 is configured to engage with the flexible segment 116 and the first anchor 104 to aid in adjusting the distance L1 between the first and second bones 108, 112. The apparatus 102 can also include a second anchor 120 configured for insertion into a second hole 122 on a first side 112a of the second bone 112. The second anchor 120 is configured to engage with the second bone 112 and the flexible segment 116.

The first anchor 104 can include any type of suture anchor, and can be manufactured from a surgical stainless steel or other suitable biocompatible material, such as 316 LVM stainless steel, titanium, and other suitable materials, such as nitinol, bio-absorbables, or non-absorbables (e.g., PEEK). First anchor 104 can also include an “all-textile” anchor (e.g., VERSALOOP™). The first anchor 104 can pass through the second hole 122 in the second bone 112 and be inserted (e.g., screwed, threaded, or pushed) within the first bone 108 in the first hole 106. The first anchor 104 can be inserted into the first bone 108 from its distal end 104b to its proximal end 104a such that the first anchor 104 does not extend all the way through the first bone 108. That is, the first hole 106 is disposed on a first side 108a (e.g., the lateral or proximal 10 side) of the first bone 108, and the first anchor 104 is configured such that a distance L2 remains between the distal end 104b of the first anchor 104 and a second side 108b (e.g., the medial or distal 12 side) of the first bone 108. In this way, no through or bore hole is required to be made in the second side 108b of the first bone 108.

The flexible segment 116 can be manufactured out of a variety of fibers or filaments including, but not limited to, polymer filaments (e.g., HMWPE, UHMWPE, PET, PTFE, PEEK, PEKK, PLA, PLLA, etc.), metallic filaments (e.g., Nitinol, Titanium, Titanium alloys, Tantalum, Stainless Steel, etc.), or organic filaments (e.g., Collagen, Silk, etc.), or other filaments such as carbon fiber or carbon nanotubes, etc., and can be made of resorbable and/or biologic materials. Flexible segment 116 can include, but is not limited to, a coreless suture, a suture with a jacket and a central core, a tape, or any other tension member available or contemplated, can be poly-coated or uncoated, and can include collagen.

The second anchor 120 can be manufactured from a surgical stainless steel or other suitable biocompatible material, such as 316 LVM stainless steel, titanium, other suitable materials, such as nitinol, bio-absorbables, or non-absorbables (e.g., PEEK). Second anchor 120 can also include an “all-textile” anchor (e.g., VERSALOOP™). Second anchor 120 can be coupled to a bone plate 202 which itself can be coupled to the first side 112a of the second bone 112. As those having skill in the pertinent art will appreciate, bone plate 202 may include one or more holes through which a physician may deliver the apparatus 102 for approximation of the first and second bones 108, 114, as further described below.

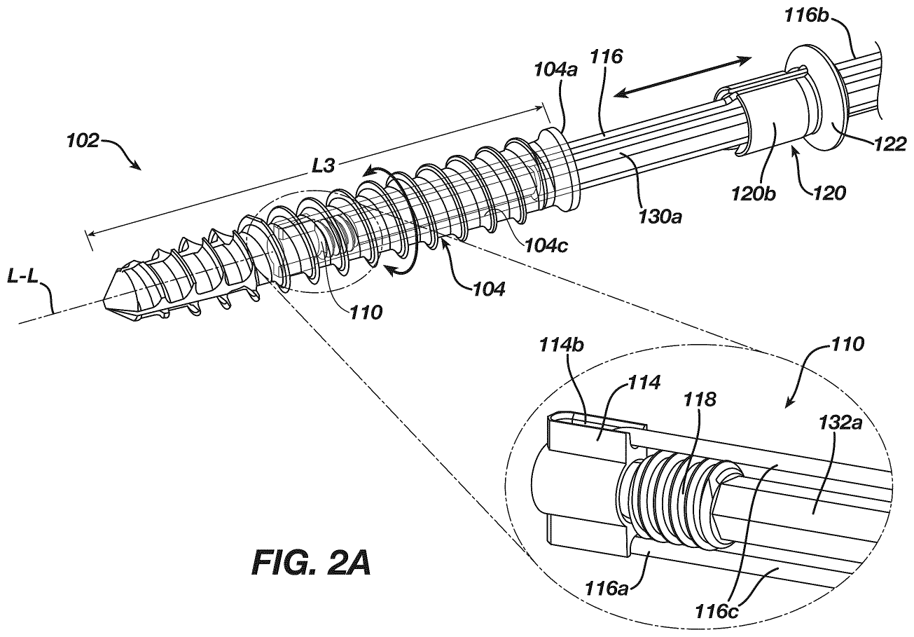

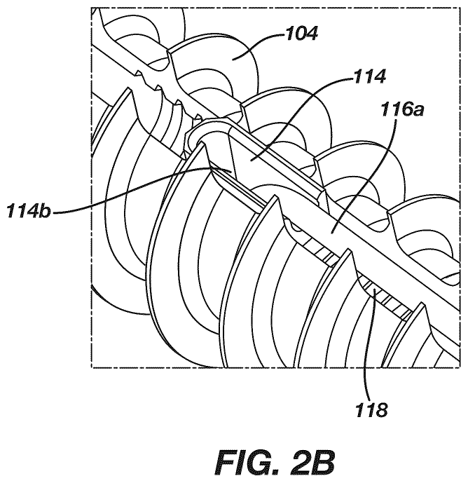

FIG. 2A is an example apparatus 102 for semi-rigid bone fixation. FIG. 2B illustrates example components of the apparatus 102 of FIG. 2A and is thus discussed simultaneously. As discussed above, the apparatus 102 can include a first anchor 104, a second anchor 120, a flexible segment 116, and an internal mechanism 110. The first anchor 104 can be internally cannulated and/or threaded along its full length. The first anchor 104 can include a lumen 104c therethrough in which the internal mechanism 110 is disposed, as further discussed below. The internal mechanism 110 includes a first component 114 (e.g., a non-rotating component) and a second component 118 (e.g., a rotating component), the first component 114 being configured to engage with one or more distal ends 116a of the flexible segment 116, and the second component 118 being configured to engage with the first component 114 and the first anchor 104 such that the second component 118 is movable along a length L3 of the first anchor 104 to cause the first component 114 to move and thereby adjust the distance L1 between the first and second bones 108, 114. In some embodiments, the second component 118 is movable along the longitudinal axis L-L of the first anchor 104. In other embodiments, the second component 118 can be offset from the longitudinal axis L-L, and still be movable along the first anchor 104.

In some embodiments, the internal mechanism 110 can be rotated and thus engaged with (e.g., screwed into) the lumen 104c of the first anchor 104, moving the internal mechanism 110 a distal direction 12 along a length of the first anchor 104 (e.g., along its longitudinal axis L-L). In some embodiments, the lumen 104c and the first component 114 are keyed to inhibit rotation of the first component 114 within the lumen 104c. In some embodiments, the first component 114 can include a groove 114b orthogonal to the longitudinal axis L-L of the first anchor 104 through which the distal end(s) 116a of the flexible segment 116 extend such that the flexible segment 116 folds proximally with its pair of extensions 116c each extending between the first component 114 and the second anchor 120.

In some embodiments, the second component 118 is configured to be threaded within the first anchor 104. The second component 118 can be configured such that as it is threaded distally into the first anchor 104, it pushes the first component 114 distally into the first anchor 104, thereby moving the distal end(s) 116a of the flexible segment 116 distally into the first anchor 104.

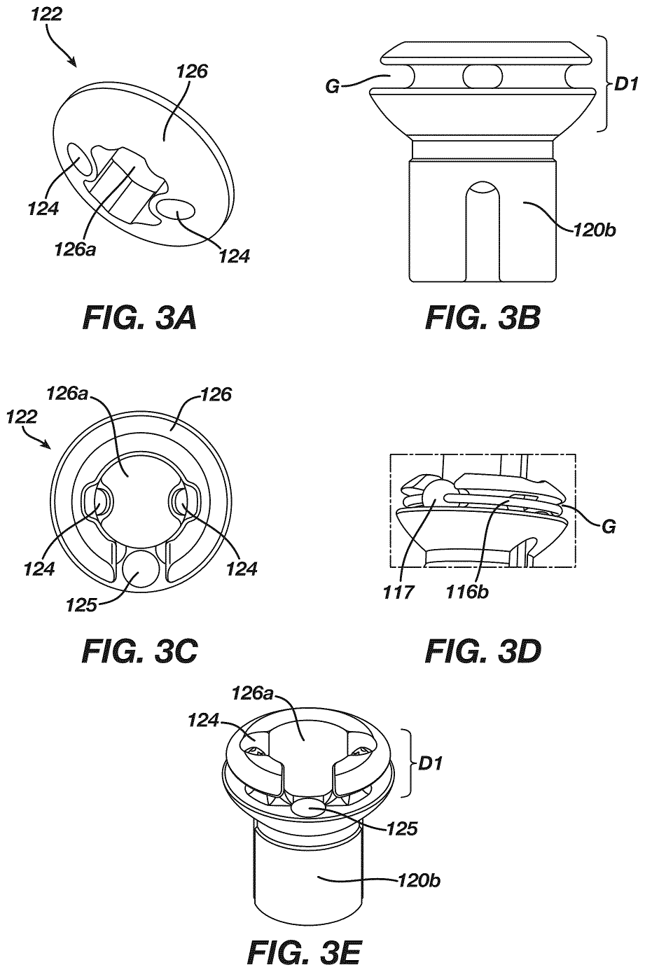

As shown in FIG. 2A, the second anchor 120 can include a cap 122 at its proximal end 120a, and a distal shaft 120b. The cap 122 is configured to engage with the first side 112a of the second bone 112, and the distal shaft 120b is configured for insertion into the second hole 122 in the second bone 112, as particularly shown in FIG. 1. FIGS. 3A-3E provide examples of how the second anchor 120 can be configured. It should be appreciated that the second anchor 120 can have a variety of shapes and/or configurations. For example, the second anchor 120 can include a cap 122 without a distal shaft 120b. The second anchor 120 can also be configured to receive and/or engage with the proximal end(s) 116b of the flexible segment 116 in a variety of ways (e.g., different types of loops, knots, etc.). For example, the cap 122 can include a button 126 having one or more openings 124 configured for receiving the one or more proximal ends 116b of the flexible segment 116, as further discussed below. The button 126 can also include a divot 125 configured for receiving a knot 117 when the proximal end(s) 116b of the flexible segment 116 are tied off or knotted, as further discussed below. The cap 122 can also be of varying thicknesses. In certain embodiments, the cap 122 can have a thickness D1 such that it can accommodate a gap or annular groove G in which the proximal end(s) 116b of the flexible segment 116 can be disposed. In some embodiments, the cap 122 can also include one or more openings 126a configured for accepting a delivery device configured to deliver apparatus 102 into the first and second bones 108, 114, as further discussed below.

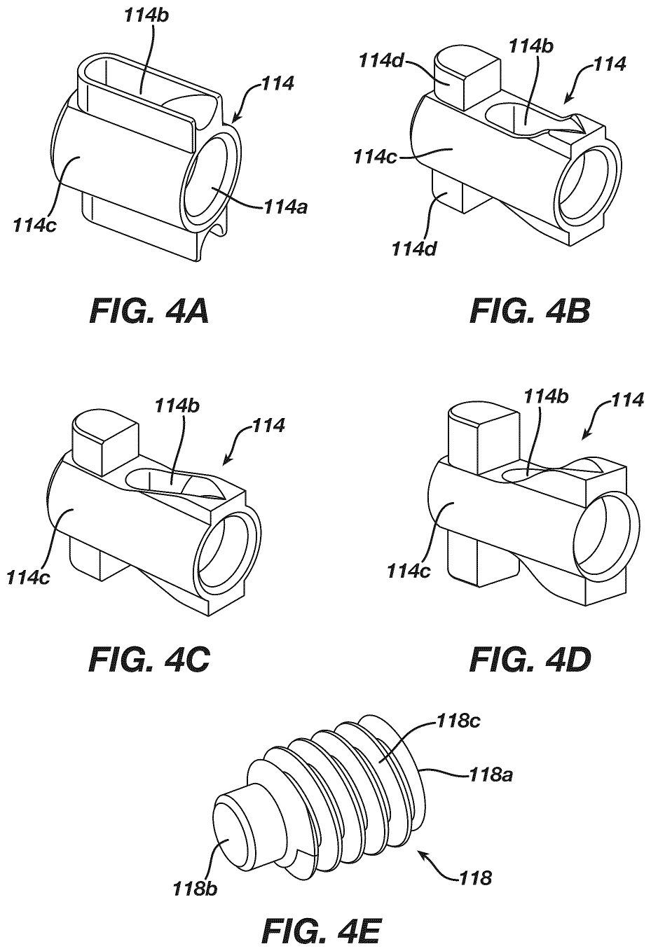

FIGS. 4A-4D provide different example first components 114 of the internal mechanism 110 of apparatus 102. As shown, first component 114 can be formed in a variety of shapes and/or configurations. For example, as shown in FIG. 4A, first component 114 can include a groove 114b that protrudes outward along the top and/or bottom of a central shaft 114c of the first component 114, as well as a proximal opening 114a (e.g., configured in a circular shape). As another example, FIG. 4B shows a first component 114 having groove 114b that sits down or is recessed within the central shaft 114c, as well as one or more protrusions 114d. FIGS. 4C and 4D show first component 114 having different shaped grooves 114b. It should be appreciated that given the different shapes and/or configurations in which first component 114 may be formed, first component can be configured to receive and/or engage with the distal end(s) 116a of the flexible segment 116 in a variety of ways (e.g., different types of loops, knots, etc.).

FIG. 4E provides an example second component 118 of the internal mechanism 110 of apparatus 102. As shown, second component 118 can have a proximal end 118a and a distal end 118b. The distal end 118b is configured to engage with the proximal opening 114a of the first component 114, while the proximal end 118a is configured to engage with a second handle shaft 132a (FIG. 2A) of a delivery device, as further discussed below. Second component 118 can also include threads 118c in such embodiments where second component 118 is configured to be threaded within the first anchor 104.

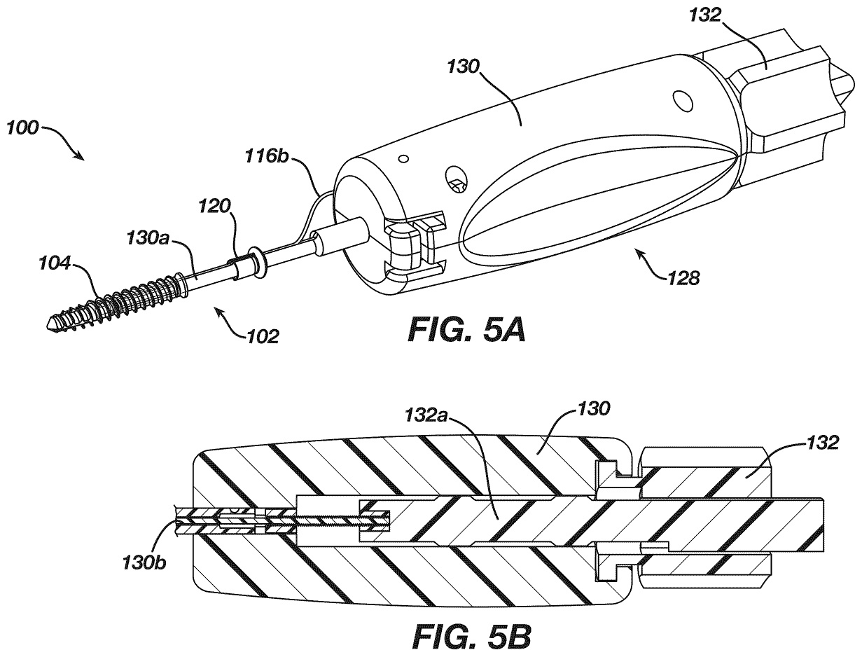

FIG. 5A is an example system 100 for the approximation of two bones (e.g., the tibia and fibula bones). The system 100 may include apparatus 102, discussed herein, and a delivery device 128. As further discussed below, delivery device 128 is configured to engage the apparatus 102 with the first and second bones 108, 112, and includes a first handle 130 and a second handle 132. The first handle 130 is configured to insert the first anchor 104 into the first bone 108, e.g., via a first rotating shaft 130a. The second handle 132 is configured to engage the internal mechanism 110 with the first anchor 104 thereby adjusting the distance L1 between the first and second bones 108, 112. The second handle 132 is also configured to engage the second anchor 120 with the second bone 112. The second handle 132 can be configured to simultaneously engage the internal mechanism 110 with the first anchor 104 while engaging the second anchor 120 with the second bone 112, as further discussed below.

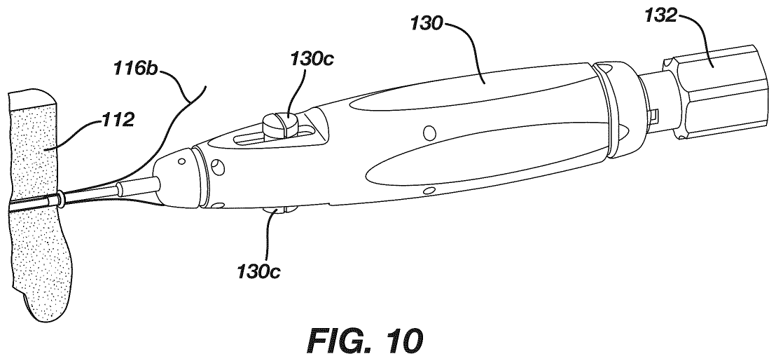

FIG. 5B shows a cross-section of the delivery device 128. As shown, the first handle 130 includes a first rotating shaft 130a with a first lumen 130b therethrough. As further discussed below, rotating the first handle 130 will rotate the first rotating shaft 130a, which will in turn rotate the first anchor 104 to engage the first anchor with the first bone 108 while maintaining a relative position of the internal mechanism 110 in relation to the first anchor 104. The second handle 132 includes a second rotating shaft 132a extending through the first lumen 130b. Rotating the second handle 132 will rotate the second rotating shaft 132a which in turn rotates a portion (e.g., the second component 118) of the internal mechanism 110 causing the internal mechanism 110 to move along a length of the first anchor 104. In some embodiments, the second anchor 120, e.g., via the cap 122, and the first rotating shaft 130a are keyed to restrict or inhibit rotation of the flexible segment 116 in relation to the cap 122 and the first anchor 104. In some embodiments, the first handle 130 is configured to engage with the proximal end(s) 116b of the flexible segment 116, such as via a retention suture post 130c (FIG. 10).

In some embodiments, as further discussed below, the first handle 130 is configured to rotate the second handle 132 without a user gripping the second handle 132. In some embodiments, the second handle 132 is removable from the first handle 130.

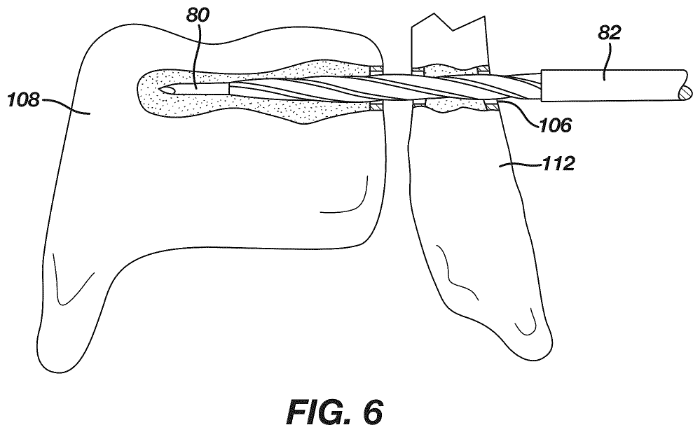

FIGS. 6-11 provide an example method for installing the apparatus 102 into two bones (e.g., the tibia and fibula bones) using the delivery device 128. It will be appreciated that the apparatus, systems, and methods described herein may also be used in other locations and/or procedures, e.g., to provide approximation between two bones other than the tibia and fibula, or between two bone fragments.

Initially, as shown in FIG. 6, a Kirschner wire, also known as a K-wire, 80 can be placed through the second bone 112 and into the first bone 108 at the appropriate location, e.g., using conventional methods, and then a drill 82 may be introduced over the Kirschner wire 80 to create the second hole 122 through the second bone 112 and at least partially into the first bone 108. The drill 82 can use a custom drill bit sized to create a clearance hole larger than four millimeters (4.0 mm), e.g., about 4.1 mm, to accommodate a first anchor 104 having an outer thread diameter of four millimeters (4.0 mm). It will be appreciated that other sizes may be provided, as desired.

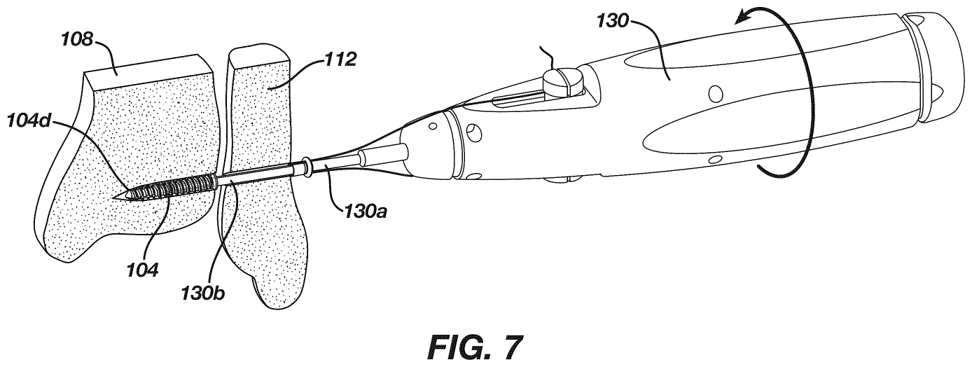

Turning to FIG. 7, the first anchor 104 may then be introduced through the clearance hole and threaded into the first bone 108 to a desired depth. For example, the first anchor 104 may be secured to a distal end 130b of the first rotating shaft 130a of the delivery device 128. Once the distal tip 104d of the first anchor 104 engages the first bone 108, the first handle 130 of the delivery device 128 may be rotated and advanced to thread the first anchor 104 into the first bone 108 to a desired depth, e.g., such that the proximal end 104a of the first anchor 104 is substantially flush with the first side 108a of the first bone 108.

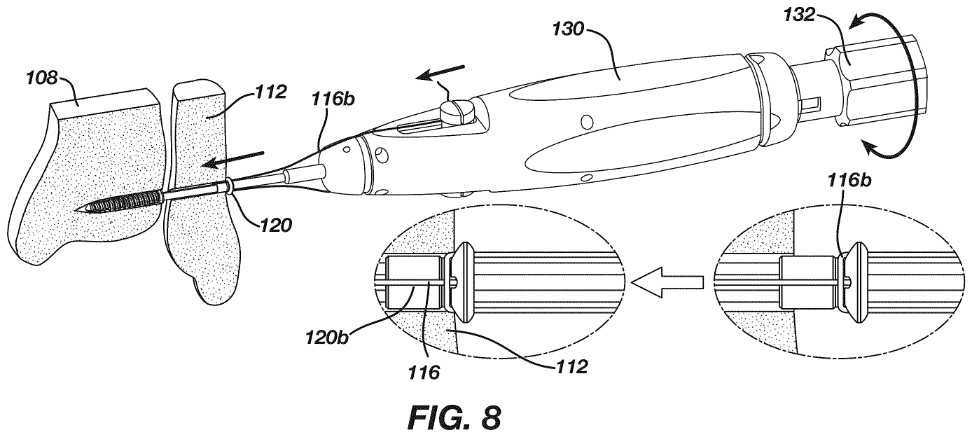

Turning to FIG. 8, once the first anchor 104 is threaded into the first bone 108 to the desired depth, the second handle 132 may be engaged with the first handle (if unattached) and rotated to insert the second anchor 120 into the second bone 112. Rotation of the second handle 132 can also simultaneously adjust placement of the internal mechanism 110 within the first anchor 104 thereby adjusting placement of the distal end 116a of the flexible segment 116 within the first anchor 104 and the distance L1 between the first and second bones 108, 112, as discussed herein.

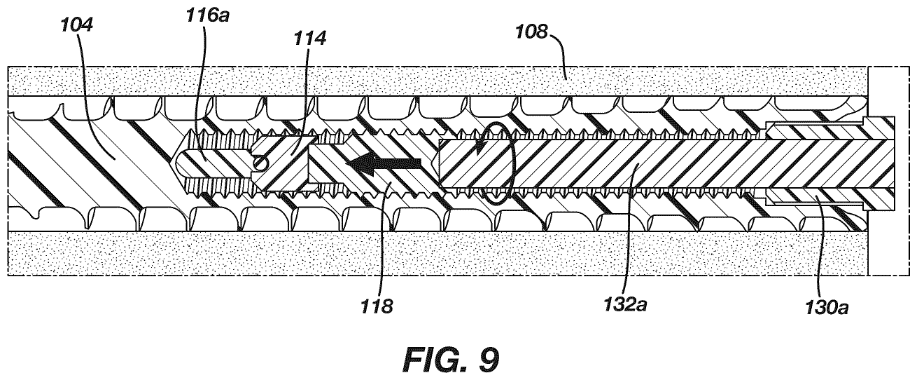

FIG. 9 provides a cross-sectional view of the first anchor 104 to show how the internal mechanism 110 is adjusted via the method step discussed above in FIG. 8. That is, as the second handle 132 is rotated, the second rotating shaft 132a (connected to the second handle 132) is rotated. Since the second rotating shaft 132a is engaged (e.g., threaded into) the second component 118 of the internal mechanism 110, the second component 118 is also driven (e.g., threaded) distally into the first anchor 104 which in turn drives (e.g., pushes) the connected first component 114—with the distal end 116 a of the flexible segment 116 attached thereto—distally into the first anchor 104. Such step provides for the flexible segment 116 being adjusted distally into the first anchor 104 and thus into the first bone 108. Since the proximal end(s) 116b of the flexible segment 116 are coupled to the second anchor 120, as discussed herein, this step allows for the adjustment of the length of the flexible segment 116 between the first and second anchors 104, 120, thereby adjusting the distance L1 between the first and second bones 108, 112.

Turning to FIG. 10, once the distance L1 between the first and second bones 108, 112 is adjusted via the above-described steps, a user (e.g., a physician) can unwind one proximal end 116b of the flexible segment 116 from the delivery device 128, e.g., via one retention suture post 130c. A second proximal end 116b can be left wound to a second retention suture post 130c on the delivery device 128.

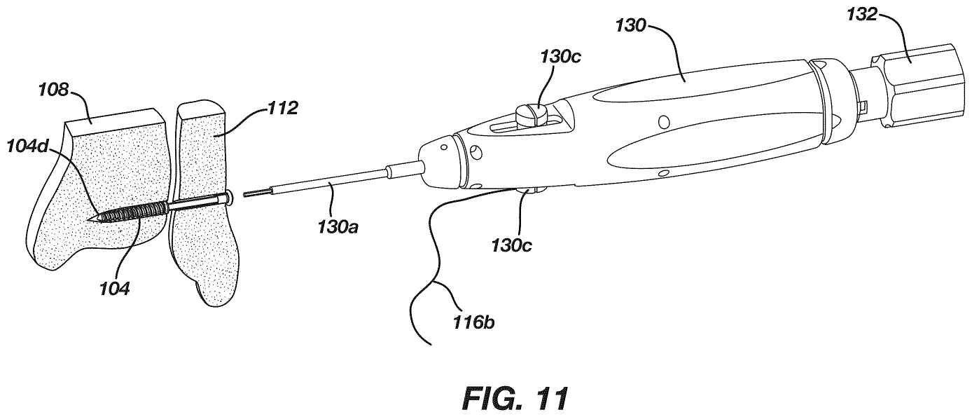

Finally, as shown in FIG. 11, the delivery device 128 can be removed, leaving the apparatus 102 engaged with the first and second bones 108, 112, as discussed herein. Once the first proximal end 116b is unwound from the first retention suture post 130c, as shown in FIG. 10, the user can then remove the delivery device 128 with the other proximal end 116b of the flexible segment 116 still attached to the delivery device 128, via the alternate (e.g., bottom) retention suture post 130c, pulling the proximal end(s) 116b through the second anchor 120 (e.g., the one or more openings 124). For example, as particularly shown in FIG. 8, the proximal end(s) 116b can be looped through the second anchor 120, e.g., via the one or more openings 124, and looped underneath the button 126 of the second anchor 120. At the same time, the proximal end(s) 116b can also be looped around and/or through the flexible segment 116. In such configuration, once the length of the flexible segment 116 is adjusted, as discussed herein, the delivery device 128 can then be removed, and the proximal end(s) 116b disengaged from the flexible segment 116 and second anchor 120, e.g., by the delivery device 128 aiding in pulling the proximal end(s) 116b away from the flexible segment 116 and through the second anchor 120.

In some examples, disclosed systems or methods may involve one or more of the following clauses:

Clause 1: An apparatus for the approximation of two bones, the apparatus comprising: a first anchor comprising a proximal end and a distal end, the first anchor being configured for insertion into a first hole in a first bone; a flexible segment; an internal mechanism configured to adjust a distance between the first bone and a second bone, the internal mechanism comprising a first component and a second component, the first component being configured to engage with a distal end of the flexible segment, and the second component being configured to engage with the first component and the first anchor such that the second component is movable along a length of the first anchor to cause the first component to move and thereby adjust the distance; and a second anchor configured to engage with the second bone and the flexible segment such that the flexible segment extends between the first component of the internal mechanism and the second anchor.

Clause 2: The apparatus of clause 1, wherein the first component comprises a groove orthogonal to a longitudinal axis of the first anchor through which the distal end of the flexible segment extends such that the flexible segment folds proximally with a pair of extensions each extending between the first component and the second anchor.

Clause 3: The apparatus of clause 1, wherein the second component is configured to be threaded within the first anchor.

Clause 4: The apparatus of clause 3, wherein the second component is configured to push the first component distally into the first anchor, thereby moving the distal end of the flexible segment distally into the first anchor as the second component is rotated distally within the first anchor.

Clause 5: The apparatus of clause 3, wherein the first anchor comprises a lumen therethrough, wherein the internal mechanism is moveable within the lumen of the first anchor, and wherein the lumen and the first component are keyed to inhibit rotation of the first component within the lumen.

Clause 6: The apparatus of clause 1, wherein the second anchor comprises: a proximal end comprising a cap configured to engage with a first side of the second bone; and a distal shaft configured for insertion into a second hole in the second bone.

Clause 7: The apparatus of clause 6, wherein the proximal end of the second anchor comprises one or more openings configured to receive one or more proximal ends of the flexible segment.

Clause 8: The apparatus of clause 1, wherein the second anchor comprises a button configured to engage with a first side of the second bone.

Clause 9: The apparatus of clause 8, wherein the button comprises one or more openings configured to receive one or more proximal ends of the flexible segment.

Clause 10: A system for the approximation of two bones, the system comprising: an apparatus comprising a first anchor, a flexible segment, an internal mechanism, and a second anchor, the first anchor comprising a distal end configured for insertion into a first hole in a first bone, the internal mechanism being configured to engage the first anchor to adjust a distance between the first bone and a second bone, the second anchor being configured to engage with the second bone, and the flexible segment extending between the internal mechanism and the second anchor; and a delivery device configured to engage the apparatus with the first and second bones, the delivery device comprising a first handle and a second handle, the first handle being configured to insert the first anchor into the first bone, and the second handle being configured to engage the internal mechanism with the first anchor, thereby adjusting the distance between the first and second bones.

Clause 11: The system of clause 10, wherein the delivery device further comprises a first rotating shaft with a lumen therethrough and a second rotating shaft extending through the lumen, wherein rotation of the first handle rotates the first rotating shaft to rotate the first anchor while maintaining a relative position of the internal mechanism in relation to the first anchor, and wherein rotation of the second handle rotates the second rotating shaft to rotate a portion of the internal mechanism to cause the internal mechanism to move along a length of the first anchor.

Clause 12: The system of clause 11, wherein the second anchor comprises a cap configured to engage with a first side of the second bone, wherein the cap comprises one or more openings configured to receive one or more proximal ends of the flexible segment, and wherein the cap and the first rotating shaft are keyed to inhibit rotation of the flexible segment in relation to the cap and the first anchor.

Clause 13: The system of clause 10, wherein the internal mechanism comprises: a first component configured to engage with a distal end of the flexible segment; and a second component configured to engage with the first component and rotate within the first anchor.

Clause 14: The system of clause 10, wherein the first handle is configured to rotate the second handle without a user gripping the second handle.

Clause 15: The system of clause 10, wherein the first handle of the delivery device is configured to engage with one or more proximal ends of the flexible segment.

Clause 16: A method for the approximation of two bones, the method comprising: delivering, via a first handle of a delivery device, a first anchor into a first bone, wherein the first anchor comprises an internal mechanism therein, and the internal mechanism engages with a distal end of a flexible segment, and wherein the flexible segment extends between the internal mechanism and a second anchor; and simultaneously, via a second handle of the delivery device: engaging the second anchor with a second bone; and adjusting placement of the internal mechanism within the first anchor thereby adjusting placement of the distal end of the flexible segment within the first anchor and a distance between the first and second bones.

Clause 17: The method of clause 16, wherein delivering the first anchor into the first bone comprises rotating the first handle to thereby rotate a first rotating shaft engaged to a proximal end of the first anchor to thereby rotate the first anchor into the first bone.

Clause 18: The method of clause 16, wherein adjusting placement of the internal mechanism within the first anchor comprises rotating the second handle to thereby rotate a second rotating shaft engaged to a threaded portion of the internal mechanism to thereby rotate the threaded portion into the first anchor.

Clause 19: The method of clause 16, wherein the internal mechanism comprises a non-rotating portion and a rotating portion, and wherein adjusting placement of the internal mechanism within the first anchor comprises inserting a distal end of the rotating portion into the non-rotating portion.

Clause 20: The method of clause 16, wherein the flexible segment comprises one or more proximal ends configured to engage with the delivery device, the method further comprising: disengaging the one or more proximal ends of the flexible segment from the delivery device; and removing the delivery device.

The descriptions contained herein are examples of embodiments of the invention and are not intended in any way to limit the scope of the invention. As described herein, the invention contemplates many variations and modifications of structures and methods, including alternative materials, alternative configurations of component parts, and alternative method steps. Modifications and variations apparent to those having skill in the pertinent art according to the teachings of this disclosure are intended to be within the scope of the claims which follow.

What is claimed is:

1. An apparatus for the approximation of two bones, the apparatus comprising:
a first anchor comprising a proximal end and a distal end, the first anchor being configured for insertion into a first hole in a first bone;
a flexible segment;
an internal mechanism configured to adjust a distance between the first bone and a second bone, the internal mechanism comprising a first component and a second component, the first component being configured to engage with a distal end of the flexible segment, and the second component being configured to engage with the first component and the first anchor such that the second component is movable along a length of the first anchor to cause the first component to move and thereby adjust the distance; and
a second anchor configured to engage with the second bone and the flexible segment such that the flexible segment extends between the first component of the internal mechanism and the second anchor.

2. The apparatus of claim 1, wherein the first component comprises a groove orthogonal to a longitudinal axis of the first anchor through which the distal end of the flexible segment extends such that the flexible segment folds proximally with a pair of extensions each extending between the first component and the second anchor.

3. The apparatus of claim 1, wherein the second component is configured to be threaded within the first anchor.

4. The apparatus of claim 3, wherein the second component is configured to push the first component distally into the first anchor, thereby moving the distal end of the flexible segment distally into the first anchor as the second component is rotated distally within the first anchor.

5. The apparatus of claim 3,
wherein the first anchor comprises a lumen therethrough,
wherein the internal mechanism is moveable within the lumen of the first anchor, and
wherein the lumen and the first component are keyed to inhibit rotation of the first component within the lumen.

6. The apparatus of claim 1, wherein the second anchor comprises:
a proximal end comprising a cap configured to engage with a first side of the second bone; and
a distal shaft configured for insertion into a second hole in the second bone.

7. The apparatus of claim 6, wherein the proximal end of the second anchor comprises one or more openings configured to receive one or more proximal ends of the flexible segment.

8. The apparatus of claim 1, wherein the second anchor comprises a button configured to engage with a first side of the second bone.

9. The apparatus of claim 8, wherein the button comprises one or more openings configured to receive one or more proximal ends of the flexible segment.

10. A system for the approximation of two bones, the system comprising:
an apparatus comprising a first anchor, a flexible segment, an internal mechanism, and a second anchor, the first anchor comprising a distal end configured for insertion into a first hole in a first bone, the internal mechanism being configured to engage the first anchor to adjust a distance between the first bone and a second bone, the second anchor being configured to engage with the second bone, and the flexible segment extending between the internal mechanism and the second anchor; and
a delivery device configured to engage the apparatus with the first and second bones, the delivery device comprising a first handle and a second handle, the first handle being configured to insert the first anchor into the first bone, and the second handle being configured to engage the internal mechanism with the first anchor, thereby adjusting the distance between the first and second bones.

11. The system of claim 10,
wherein the delivery device further comprises a first rotating shaft with a lumen therethrough and a second rotating shaft extending through the lumen,
wherein rotation of the first handle rotates the first rotating shaft to rotate the first anchor while maintaining a relative position of the internal mechanism in relation to the first anchor, and
wherein rotation of the second handle rotates the second rotating shaft to rotate a portion of the internal mechanism to cause the internal mechanism to move along a length of the first anchor.

12. The system of claim 11,
wherein the second anchor comprises a cap configured to engage with a first side of the second bone,
wherein the cap comprises one or more openings configured to receive one or more proximal ends of the flexible segment, and
wherein the cap and the first rotating shaft are keyed to inhibit rotation of the flexible segment in relation to the cap and the first anchor.

13. The system of claim 10, wherein the internal mechanism comprises:
a first component configured to engage with a distal end of the flexible segment; and
a second component configured to engage with the first component and rotate within the first anchor.

14. The system of claim 10, wherein the first handle is configured to rotate the second handle without a user gripping the second handle.

15. The system of claim 10, wherein the first handle of the delivery device is configured to engage with one or more proximal ends of the flexible segment.

16. A method for the approximation of two bones, the method comprising:
delivering, via a first handle of a delivery device, a first anchor into a first bone,
wherein the first anchor comprises an internal mechanism therein, and the internal mechanism engages with a distal end of a flexible segment, and
wherein the flexible segment extends between the internal mechanism and a second anchor; and
simultaneously, via a second handle of the delivery device:
engaging the second anchor with a second bone; and
adjusting placement of the internal mechanism within the first anchor thereby adjusting placement of the distal end of the flexible segment within the first anchor and a distance between the first and second bones.

17. The method of claim 16, wherein delivering the first anchor into the first bone comprises rotating the first handle to thereby rotate a first rotating shaft engaged to a proximal end of the first anchor to thereby rotate the first anchor into the first bone.

18. The method of claim 16, wherein adjusting placement of the internal mechanism within the first anchor comprises rotating the second handle to thereby rotate a second rotating shaft engaged to a threaded portion of the internal mechanism to thereby rotate the threaded portion into the first anchor.

19. The method of claim 16, wherein the internal mechanism comprises a non-rotating portion and a rotating portion, and wherein adjusting placement of the internal mechanism within the first anchor comprises inserting a distal end of the rotating portion into the non-rotating portion.

20. The method of claim 16, wherein the flexible segment comprises one or more proximal ends configured to engage with the delivery device, the method further comprising:
disengaging the one or more proximal ends of the flexible segment from the delivery device; and
removing the delivery device.
